(19) United States
(12) Patent Application Publication
(10) Pub. No.: US 2021/0118524 A1
(43) Pub. Date: Apr. 22, 2021

(54) Identifying Therapeutic Targets by Discovering Interactions between Non-Coding Variants and Candidate Genes

(71) Applicant: Camp4 Therapeutics Corporation, Cambridge, MA (US)

(72) Inventors: Yuting Liu, Brookline, MA (US); Asher Schachter, Needham, MA (US); Alla Sigova, Newton, MA (US); David A. Bumcrot, Belmont, WA (US); Iris Grossman, Brookline, MA (US)

(21) Appl. No.: 17/072,283

(22) Filed: Oct. 16, 2020

Related U.S. Application Data
(60) Provisional application No. 62/915,946, filed on Oct. 16, 2019.

(51) Int. Cl. G16B 15/30; G16B 20/20

(52) U.S. Cl. CPC G16B 15/30; G16B 20/20

(57) ABSTRACT

Disclosed herein are methods for linking non-coding variants and candidate genes that are associated with one another through long-range chromatin interactions. Thus, these non-coding variants may be involved in the expression of a candidate gene and therefore, serve as possible therapeutic targets for treating diseases in which the expression fo the candidate gene is dysreguated. A non-coding variant can be linked to a candidate gene by analyzing datasets including chromatin accessibility data (e.g., ATAC-seq data), protein-chromatin binding site pairing data, and/or chromatin contact profile. For example, a non-coding variant can be linked to a candidate gene by identifying an enhancer-promoter loop through a long range chromatin interaction. As another example, a non-coding variant can be linked to a candidate gene through a statistical eQTL analysis. Altogether, such links between non-coding variants and candidate genes can be used to identify novel disease genes and relevant therapies for treating related diseases.

### CROSS REFERENCE TO RELATED APPLICATIONS

This application claims the benefit of and priority to U.S. Provisional Application No. 62/915,946, filed Oct. 16, 2019, which is incorporated herein by reference in its entirety for all purposes.

### SUMMARY OF THE INVENTION

Disclosed herein are methods for identifying therapeutic targets by discovering interactions between non-coding variants and candidate genes.

Disclosed herein is a method, comprising: obtaining a dataset comprising: sequencing or genotyping data comprising a non-coding variant (NCV); chromatin accessibility data identifying chromatin-accessible regions across the genome; and chromatin contact profile describing chromatin-chromatin interactions and chromatin domains; identifying that the NCV is located in a first chromatin-accessibility region representing a regulatory element; identifying a candidate gene whose expression is dependent upon the regulatory element; determining that at least a threshold number of chromatin-chromatin interactions exist between a location of the first chromatin-accessible region representing the regulatory element and the location of the second chromatin-accessible region representing a promoter of the candidate gene, thereby indicating that the promoter of the candidate gene and the regulatory element are physically linked through a long range chromatin interaction; and linking the candidate gene to the NCV in the linkage disequilibrium block. In various embodiments, the obtained dataset further comprises protein—chromatin binding site pairing data, and wherein identifying that the NCV is located in a first chromatin-accessibility region representing a regulatory element comprises determining that the regulatory element is positioned at the first chromatin-accessibility region using the protein-chromatin binding site pairing data. In various embodiments, the regulatory element is an active enhancer.

In various embodiments, determining that the regulatory element is positioned at the first chromatin-accessibility region using the protein-chromatin binding site pairing data comprises identifying the regulatory elements as an active enhancer as opposed to a promoter by comparing the chromatin accessibility data to the protein—chromatin binding site pairing data. In various embodiments, the chromatin accessibility data corresponding to the regulatory element comprises a peak indicating presence of a regulatory element, and wherein the protein-chromatin binding site pairing data corresponding to the regulatory element lacks a peak, thereby indicating lack of a promoter. In various embodiments, identifying a candidate gene whose expression is dependent upon the regulatory element comprises: determining a location of a promoter of the candidate gene based on a location of a second chromatin-accessible region in the chromatin accessibility data; and constructing an enhancer-promoter (E-P) loop comprising the regulatory element and the promoter of the candidate gene.

In various embodiments, the dataset further comprises a genetic variant, wherein identifying a candidate gene whose expression is dependent upon the regulatory element comprises identifying the candidate gene as a gene whose expression is affected by the genetic variant. In various embodiments, the genetic variant is selected from any one of an expression quantitative trait loci (eQTL) variant determined from a meta-analysis of Genotype Tissue Expression (GTEx) data or a source of gene expression profile data measured with DNA sequence variation data. In various embodiments, the protein-chromatin binding site pairing data comprises ChIP-seq data. In various embodiments, the sequencing or genotyping data comprising the non-coding variant (NCVs) is Genome-Wide Association Study (GWAS) data. In various embodiments, the sequencing or genotyping data comprising the non-coding variant (NCVs) is obtained from whole genome sequencing, whole exome sequencing, targeted of a gene region or panel of genes, or targeted genotyping.

In various embodiments, the chromatin accessibility data is Assay for Transposase-Accessible Chromatin (ATAC) sequencing data comprising ATAC peaks that identify the chromatin-accessible regions across the genome. In various embodiments, the chromatin contact profile is HiChIP-seq data comprising HiChIP paired-end-tags (PETs) describing the chromatin-chromatin interactions. In various embodiments, the threshold number of chromatin-chromatin interactions is represented by three, four, five, six, seven, or eight HiChIP paired-end-tags. In various embodiments, at least one of the chromatin-chromatin interactions of the chromatin contact profile is an enhancer-promoter interaction. In various embodiments, at least one of the chromatin domains of the chromatin contact profile is an insulated neighborhood.

In various embodiments, the dataset further comprises transcription factor binding motif data, wherein the method further comprises: using the transcription factor binding motif data, identifying a target protein that binds at the location of the first chromatin-accessible region representing the regulatory element; determining a signaling pathway that the target protein is involved in; selecting one or more therapeutics capable of modulating the signaling pathway which would subsequently modulate expression of the candidate gene. In various embodiments, using the transcription factor binding motif data comprises identifying transcription factor binding motif data corresponding to sequences located between the first chromatin-accessible region and the second chromatin-accessible region. In various embodiments, the one or more therapeutics capable of modulating the signaling pathway target one of the target protein or a receptor in the signaling pathway.

In various embodiments, the target protein is STAT1 and wherein the signaling pathway is a JAK-STAT signaling pathway. In various embodiments, the receptor in the signaling pathway is an Interferon Gamma (IFNG) receptor. In various embodiments, the candidate gene is CTSS. In various embodiments, the target protein is SMAD3 and wherein the signaling pathway is a transforming growth factor beta (TGFB) signaling pathway. In various embodiments, the receptor in the signaling pathway is a TGFB receptor. In various embodiments, the candidate gene is SLC41A1.

In various embodiments, the dataset further comprises pairwise linkage disequilibrium measurements. In various embodiments, identifying that the NCV is located in a first chromatin-accessibility region representing a regulatory element comprises determining that a linkage disequilibrium block determined from the pairwise linkage disequilibrium measurements comprises the NCV and the first chromatin-accessible region representing a regulatory element. In various embodiments, the linkage disequilibrium block is based on pairwise linkage disequilibrium measurements where R²>0.8. In various embodiments, the linkage disequilibrium block is determined from the pairwise linkage disequilibrium measurements using a Plink algorithm. In various embodiments, the candidate gene is linked to the NCV through an insulated neighborhood.

In various embodiments, the chromatin accessibility data is human, cell-specific chromatin accessibility data. In various embodiments, the chromatin contact profile is human, cell-specific chromatin contact profile data. In various embodiments, the NCVs are across a genome, within a region of the genome, or within a chromosome. In various embodiments, the NCV linked to the candidate gene is linked to a clinically, pharmacologically, or phenotypically relevant human attribute. In various embodiments, the NCV linked to the candidate gene is linked to a disease-related human attribute.

Additionally disclosed herein is a non-transitory computer readable medium comprising instructions that, when executed by a processor, cause the processor to: obtain a dataset comprising: sequencing or genotyping data comprising a non-coding variant (NCV); chromatin accessibility data identifying chromatin-accessible regions across the genome; and chromatin contact profile describing chromatin-chromatin interactions and chromatin domains; identify that the NCV is located in a first chromatin-accessibility region representing a regulatory element; identify a candidate gene whose expression is dependent upon the regulatory element; determine that at least a threshold number of chromatin-chromatin interactions exist between a location of the first chromatin-accessible region representing the regulatory element and the location of the second chromatin-accessible region representing a promoter of the candidate gene, thereby indicating that the promoter of the candidate gene and the regulatory element are physically linked through a long range chromatin interaction; and link the candidate gene to the NCV in the linkage disequilibrium block. In various embodiments, the dataset further comprises protein-chromatin binding site pairing data, and wherein the instructions that cause the processor to identify that the NCV is located in a first chromatin-accessibility region representing a regulatory element further comprises instructions that, when executed, cause the processor to determine that the regulatory element is positioned at the first chromatin-accessibility region using the protein-chromatin binding site pairing data.

In various embodiments, the regulatory element is an active enhancer. In various embodiments, the instructions that cause the processor to determine that the regulatory element is positioned at the first chromatin-accessibility region using the protein-chromatin binding site pairing data further comprises instructions that, when executed by the processor, cause the processor to identify the regulatory elements as an active enhancer as opposed to a promoter by comparing the chromatin accessibility data to the protein—chromatin binding site pairing data. In various embodiments, the chromatin accessibility data corresponding to the regulatory element comprises a peak indicating presence of a regulatory element, and wherein the protein-chromatin binding site pairing data corresponding to the regulatory element lacks a peak, thereby indicating lack of a promoter.

In various embodiments, the instructions that cause the processor to identify a candidate gene whose expression is dependent upon the regulatory element further comprises instructions that, when executed by the processor, cause the processor to: determine a location of a promoter of the candidate gene based on a location of a second chromatin-accessible region in the chromatin accessibility data; and construct an enhancer-promoter (E-P) loop comprising the regulatory element and the promoter of the candidate gene.

In various embodiments, the dataset further comprises a genetic variant, wherein the instructions that cause the processor to identify a candidate gene whose expression is dependent upon the regulatory element further comprises instructions that, when executed by the processor, cause the processor to identify the candidate gene as a gene whose expression is affected by the genetic variant. In various embodiments, the genetic variant is selected from any one of an expression quantitative trait loci (eQTL) variant determined from a meta-analysis of Genotype Tissue Expression (GTEx) data or a source of gene expression profile data measured with DNA sequence variation data.

In various embodiments, the protein-chromatin binding site pairing data comprises ChIP-seq data. In various embodiments, the sequencing or genotyping data comprising the non-coding variant (NCVs) is Genome-Wide Association Study (GWAS) data. In various embodiments, the sequencing or genotyping data comprising the non-coding variant (NCVs) is obtained from whole genome sequencing, whole exome sequencing, targeted of a gene region or panel of genes, or targeted genotyping. In various embodiments, the chromatin accessibility data is Assay for Transposase-Accessible Chromatin (ATAC) sequencing data comprising ATAC peaks that identify the chromatin-accessible regions across the genome. In various embodiments, the chromatin contact profile is HiChIP-seq data comprising HiChIP paired-end-tags (PETs) describing the chromatin-chromatin interactions. In various embodiments, the threshold number of chromatin-chromatin interactions is represented by three, four, five, six, seven, or eight HiChIP paired-end-tags.

In various embodiments, at least one of the chromatin-chromatin interactions of the chromatin contact profile is an enhancer-promoter interaction. In various embodiments, at least one of the chromatin domains of the chromatin contact profile is an insulated neighborhood. In various embodiments, the dataset further comprises transcription factor binding motif data, wherein the instructions further comprise instructions that, when executed by the processor, cause the processor to: use the transcription factor binding motif data, identifying a target protein that binds at the location of the first chromatin-accessible region representing the regulatory element; determine a signaling pathway that the target protein is involved in; select one or more therapeutics capable of modulating the signaling pathway which would subsequently modulate expression of the candidate gene. In various embodiments, the instructions that cause the processor to use the transcription factor binding motif data further comprises instructions that, when executed by the processor, cause the processor to identify transcription factor binding motif data corresponding to sequences located between the first chromatin-accessible region and the second chromatin-accessible region.

In various embodiments, the one or more therapeutics capable of modulating the signaling pathway target one of the target protein or a receptor in the signaling pathway. In various embodiments, the target protein is STAT1 and wherein the signaling pathway is a JAK-STAT signaling pathway. In various embodiments, the receptor in the signaling pathway is an Interferon Gamma (IFNG) receptor. In various embodiments, the candidate gene is CTSS. In various embodiments, the target protein is SMAD3 and wherein the signaling pathway is a transforming growth factor beta (TGFB) signaling pathway. In various embodiments, the receptor in the signaling pathway is a TGFB receptor. In various embodiments, the candidate gene is SLC41A1.

In various embodiments, the dataset further comprises pairwise linkage disequilibrium measurements. In various embodiments, the linkage disequilibrium block is based on pairwise linkage disequilibrium measurements where R²>0.8. In various embodiments, the linkage disequilibrium block is determined from the pairwise linkage disequilibrium measurements using a Plink algorithm.

In various embodiments, the candidate gene is linked to the NCV through an insulated neighborhood. In various embodiments, the chromatin accessibility data is human, cell-specific chromatin accessibility data. In various embodiments, the chromatin contact profile is human, cell-specific chromatin contact profile data. In various embodiments, the NCVs are across a genome, within a region of the genome, or within a chromosome. In various embodiments, the NCV linked to the candidate gene is associated with a clinically, pharmacologically, or phenotypically relevant human attribute. In various embodiments, the NCV linked to the candidate gene is associated with a disease-related human attribute.

### BRIEF DESCRIPTION OF THE SEVERAL VIEWS OF THE DRAWINGS

These and other features, aspects, and advantages of the present invention will become better understood with regard to the following description, and accompanying drawings, where:

Figure (FIG. 1 depicts a flow diagram for discovering interactions between non-coding variants and genes.

FIG. 2A depicts an example set of data including chromatin-accessibility data and protein-chromatin binding site pairing data corresponding to an active enhancer.

FIG. 2B depicts an example set of data including chromatin-accessibility data and protein-chromatin binding site pairing data corresponding to a promoter.

FIG. 3 depicts an example diagram for identifying interactions between a non-coding variant and a target gene.

FIG. 4A depicts an example linkage between a non-coding variant and a target gene CTSS.

FIG. 4B depicts an example linkage between a non-coding variant and a target gene SLC41A1.

FIG. 5A depicts an overall pipeline of genes that were linked to activated enhancers using the methods described herein.

FIG. 5B depicts overlapping non-coding variant and gene pairs using the EP loop method or the genetic association method.

FIG. 5C depicts examples linkages between trait-associated non-coding variants and target genes.

FIG. 6 illustrates an example computer for implementing the entities shown in FIG. 1.

### DETAILED DESCRIPTION

### Definitions

The term “obtaining a dataset” encompasses obtaining a set of data determined from at least one sample. Obtaining a dataset encompasses obtaining a sample and processing the sample to experimentally determine the data (e.g., performing one or more assays to determine the data). The phrase also encompasses creating a dataset. The phrase also encompasses receiving a set of data, e.g., from a third party that has processed the sample to experimentally determine the dataset. Additionally, the phrase encompasses mining data from at least one database or at least one publication or a combination of databases and publications. A dataset can be obtained by one of skill in the art via a variety of known ways including stored on a storage memory. In various embodiments, as described herein, a dataset can include one or more of sequencing or genotyping data comprising a non-coding variant (NCV), chromatin accessibility data identifying chromatin-accessible regions across the genome, chromatin contact profile describing chromatin-chromatin interactions and chromatin domains, and/or protein—chromatin binding site pairing data.

The phrase “non-coding variant” refers to one or more nucleotide bases in a region of nucleic acid that does not encode for protein sequences. A non-coding variant refers to one or more nucleotide bases that differ from a known reference base (e.g., reference base of a reference genome sequence or reference base determined from sequences obtained from a control cell). For example, a non-coding variant can be a single nucleotide polymorphism. Although a non-coding variant does not encode for protein sequences, it may still be implicated in the regulation of a gene, as is determined using the methods described herein. In various embodiments, a non-coding variant can be a trait-associated non-coding variant that is linked to a disease or trait such that it causes a higher risk for the disease or trait.

Any terms not directly defined herein shall be understood to have the meanings commonly associated with them as understood within the art of the disclosure. Certain terms are discussed herein to provide additional guidance to the practitioner in describing the compositions, devices, methods and the like of aspects of the disclosure, and how to make or use them. It will be appreciated that the same thing can be said in more than one way. Consequently, alternative language and synonyms can be used for any one or more of the terms discussed herein. No significance is to be placed upon whether or not a term is elaborated or discussed herein. Some synonyms or substitutable methods, materials and the like are provided. Recital of one or a few synonyms or equivalents does not exclude use of other synonyms or equivalents, unless it is explicitly stated. Use of examples, including examples of terms, is for illustrative purposes only and does not limit the scope and meaning of the aspects of the disclosure herein.

Additionally, as used in the specification, the singular forms “a,” “an” and “the” include plural referents unless the context clearly dictates otherwise.

Overview

Disclosed herein are methods and non-transitory computer readable media for identifying therapeutic targets by discovering long range chromatin interactions between non-coding variants and genes.

In various embodiments, the methods and non-transitory computer readable media describe the analysis of a dataset to identify non-coding variants associated with enhancer-promoter loops (EP loop), thereby enabling the linking between non-coding variant and a candidate gene whose expression is controlled by a promoter of the EP loop.

In various embodiments, the methods and non-transitory computer readable media describe the analysis of a dataset to identify linkage disequilibrium blocks that include non-coding variants associated with complex traits or disease, based on GWAS analysis, known variants that affect expression of genes (e.g., based on eQTL analysis), and a chromatin region corresponding to regulatory elements (e.g., an enhancer). The trait-associated non-coding variant in the linkage disequilibrium block is identified to be linked to a promoter of the candidate gene, the expression of which is associated with a known variant (termed eQTL marker/variant). Furthermore, there is linkage disequilibrium between the trait-associated NCV and an open chromatin region that marks an enhancer of the candidate gene (identified by ATAC-seq), as evidenced by chromatin interactions between the gene and the enhancer (identified by HiChIP-seq or similar method).

Altogether, the linking of a non-coding variant to a candidate gene indicates that the non-coding variant likely affects expression of the candidate gene through disruption of transcriptional regulation and long range chromatin interactions (e.g., in an insulated neighborhood). Such links between trait-associated non-coding variants and candidate genes can be used to identify novel disease genes and thus relevant therapies for treating these or related diseases.

Additionally, the description below generally describes hepatocyte related traits in reference to trait-associated NCVs. Examples of hepatocyte related traits include but are not limited to: cholesterol metabolism, cholesterol transport, fatty acid degradation, triglyceride homeostasis, reverse cholesterol transport, and cholesterol (e.g., low-density lipoproteins (LDL) or high-density lipoproteins (HDL)). In various embodiments, the methods described herein can be useful for assigning candidate genes to other trait-associated NCVs (e.g., non-hepatocyte trait-associated NCVs). As an example, the methods described herein can be useful for analyzing data derived from non-hepatocytes (e.g., brain cells such as microglia) for assigning candidate genes to microglia trait-associated NCVs.

I. Methods

The methods disclosed herein include analyzing a dataset including data pertaining to DNA sequences, epigenomic, and chromatin characteristics to predict three-dimensional chromatin structures and active enhancers, which include long range chromatin interactions between non-coding variants (NCVs) and enhancer regions across the genome and the promoter of candidate genes. The identified interactions between NCVs and candidate genes can be leveraged to identify therapeutic targets for treatment of diseases.

FIG. 1 depicts a flow diagram for identifying long range chromatin interactions between non-coding variants and genes. At step 110, a dataset is obtained. In various embodiments, the dataset includes the following data: sequencing/genotyping data, one or more genetic variants, linkage disequilibrium data, chromatin accessibility data (e.g., from ATAC-seq), chromatin contact profile data (e.g., from HiChIP-seq), protein-chromatin binding site data (e.g., from ChIP-seq), and DNA-binding motif data (e.g., transcription factor binding motif data). In various embodiments, the dataset includes additional or fewer data. For example, the dataset can include: sequencing/genotyping data that includes trait-associated non coding variants (NCVs), chromatin accessibility data (e.g., from ATAC-seq), chromatin contact profile data (e.g., from HiChIP-seq), and protein-chromatin binding site data (e.g., from ChIP-seq).

In various embodiments, the sequencing/genotyping data includes data obtained from a Genomewide Association Study (GWAS) which associates non-coding variants (NCVs) with human traits, including diseases, or other properties of the genome, including gene expression. In some embodiments, the sequencing/genotyping data is across a genome, within any region of the genome, or corresponds to a chromosome. In various embodiments, the sequencing data can include a genetic variant that is known to affect the expression of a gene. In some embodiments, at least one of the NCVs is associated with a clinically, pharmacologically, or phenotypically relevant human attribute. In some embodiments, at least one of the NCVs is associated with a disease-related human attribute. In some embodiments, the sequencing/genotyping data comprises non coding variants (NCVs) across the genome that are obtained from whole genome sequencing, whole exome sequencing, targeted of a gene region or panel of genes, or targeted genotyping.

In various embodiments, the one or more genetic variants are included in a expression quantified trait loci (eQTL) dataset. In various embodiments, the genetic variants are non-coding variants. In various embodiments, genetic variants are eQTL variants. In various embodiments, the eQTL dataset can be generated via methods such as through a meta-analysis of Genotype-Tissue Expression (GTEx) data and one or more additional eQTL studies. An example of such an analysis is described in Strunz et al., “A mega-analysis of expression quantitative trait loci (eQTL) provides insight into the regulatory architecture of gene expression variation in liver,” Scientific Reports, volume 8, Article number: 5865 (2018), which is hereby incorporated by reference in its entirety. In various embodiments, the additional eQTL studies are conducted in the human liver. In various embodiments, the sequencing data can be curated from publicly available datasets. In various embodiments, the eQTL dataset represents gene expression profile data measured with DNA sequence variation.

In various embodiments, the linkage disequilibrium (LD) data includes pairwise LD data between pairs of markers (e.g., alleles). In various embodiments, the LD data further includes a pairwise LD measure (e.g., a LD measure D, or a square of a coefficient correlation R²) that indicates the strength of linkage between the pair of markers in one or more human populations. A neighboring group of variants with pairwise LD measure >0.8 comprise an LD block.

In various embodiments, the chromatin accessibility data represents data that elucidates DNA-protein interaction sites for transcription factors and chromatin binding proteins. For example, the chromatin accessibility data may identify open chromatin regions on a chromatin occupied by regulatory elements such as an enhancer, repressor, or a promoter. In various embodiments, the chromatin accessibility data is obtained by performing a DNase-seq assay. In various embodiments, the chromatin accessibility data is obtained by performing a Formaldehyde-Assisted Isolation of Regulatory Elements (FAIRE)-seq assay. In various embodiments, the chromatin accessibility data is obtained by performing an Assay for Transposase-Accessible Chromatin using sequencing (ATAC-seq) technique which assesses genome-wide chromatin accessibility. Further details of these techniques for obtaining chromatin accessibility data is described in Tsompana et al., Chromatin Accessibility: a window into the genome, Epigenetics & Chromatin 7, Article number: 33 (2014), which is hereby incorporated by reference in its entirety. In various embodiments, the chromatin accessibility data is relevant for a specific human cell-type. In various embodiments, the chromatin accessibility data identifies chromatin-accessible regions across the human genome, in a cell-type specific setting. In various embodiments, the chromatin-accessible regions corresponding to regulatory elements include one or more non-coding variants.

In various embodiments, the chromatin contact profile data describes chromatin domains and chromatin-chromatin interactions (e.g., DNA-DNA interactions). Chromatin-chromatin interactions, in some embodiments, are mediated by SMC1A, CTCF and H3K27Ac. The chromatin contact profile data enables profiling of three dimensional chromatin structures. In various embodiments, one or more of the chromatin-chromatin interactions described in the chromatin contact profile data are enhancer-promoter interactions. In various embodiments, one or more of the chromatin domains described in the chromatin contact profile data are insulated neighborhoods. In various embodiments, the chromatin contact profile data is obtained by performing Hi-Chromatin Immunopreciptation (HiChIP) followed by sequencing (e.g., next generation sequencing). In various embodiments, HiChIP-seq includes performing paired-end-tag (PET) sequencing for ultra-high-throughput sequencing. In various embodiments, the chromatin contact profile data is relevant for a specific human cell-type. In various embodiments, the chromatin contact profile data is HiChIP PETs describing chromatin-chromatin interactions across the human genome in a cell-type specific manner.

In various embodiments, the protein-chromatin binding site data represents data that reveals binding sites of transcription factors as well as the chromatin binding proteins (e.g., transcription factors) and histone modifications that bind to those particular binding sites. The protein-chromatin binding site paring data may further include data that reveals histone modifications (e.g., H3K4me3 which is an epigenetic modification to the DNA packaging protein Histone H3). In various embodiments, the protein-chromatin binding site data is obtained by performing Chromatin Immunoprecipitation (ChIP) followed by sequencing (e.g., next generation sequencing).

In various embodiments, the DNA-binding motif data represents data that describes particular DNA sequences. For example, the DNA-binding motif data may describe transcription factor binding motifs (e.g., specific DNA sequences where transcription factors bind to the chromatin). In various embodiments, the chromatin motif is obtained through publicly available databases (e.g., Human Transcription Factor motifs database).

Steps 120, 130, 140, and 150 generally describe a process for assigning a candidate gene to a trait-associated NCV using the dataset obtained in step 110. In various embodiments, the steps 120, 130, 140, and 150 describe a process that analyzes enhancer-promoter loops (EP loops) using the dataset obtained in step 110 (hereafter referred to as the EP loop method). In various embodiments, the steps 120, 130, 140, and 150 describe a process that analyzes associations between genomic variants and gene expression (hereafter referred to as the genetic association method). In various embodiments, steps 120, 130, 140, and 150 can involve performing both of the EP loop method and the genetic association method. Thus, the results determined from the EP loop method and the genetic association method can be validated against one another to confirm candidate genes that are to be assigned to a trait-associated NCV.

At step 120, a trait-associated NCV located in a first chromatin-accessibility region representing an activated regulatory element is identified.

Referring to the EP loop method, in various embodiments, identifying a trait-associated NCV located in a first chromatin-accessibility region representing a regulatory element involves comparing the sequencing/genotyping data to the chromatin-accessibility data to identify a regulatory element. For example, for a position at which the NCV is located, the corresponding position in the chromatin-accessibility data is analyzed to determine whether a regulatory element is present at the corresponding location. In various embodiments, presence of a regulatory element is indicated by the presence of a peak in the chromatin-accessibility data. Examples of a regulatory element include a promoter, a repressor, or an enhancer. Therefore, the presence of a NCV in a regulatory element could lead to changes in the expression of a gene that is controlled by the regulatory element.

Referring to the genetic association method, in various embodiments, identifying a trait-associated NCV located in a first chromatin-accessibility region representing an activated regulatory element involves identifying a LD block. A LD block is identified using the pairwise LD measurements. In various embodiments, a LD block is generated from pairwise LD measurements with a R² correlation coefficient greater than a threshold value. In various embodiments, the threshold value is 0.5, 0.6, 0.7, 0.8, 0.9, or 0.95. In various embodiments, a LD block is generated using an algorithm such as Plink, which measures the pairwise LD of multiple variants across the genome. In various embodiments, the LD block includes 1) a GWAS trait-associated NCV (as identified in the sequencing/genotyping data) 2) a genetic variant, and 3) and a first chromatin-accessible region representing a regulatory element. In various embodiments, the genetic variant is a NCV or a coding variant associated with expression of a gene in the same insulated neighborhood as the GWAS trait-associated NCV. In some embodiments, the LD block may include more than one GWAS trait-associated NCV, more than one genetic variant (e.g., eQTL NCV), and/or more than one chromatin-accessible region (e.g., regulatory region). In various embodiments, at step 120, the genetic association method involves identifying a non-coding variant that is located within proximity to a genetic variant and need not implement LD blocks. In various embodiments, a non-coding variant is located within proximity to a genetic variant if the non-coding variant is less than 100 nucleotide bases from the genetic variant.

At step 130, one or more candidate genes are identified. Here, a candidate gene refers to a gene whose expression is dependent upon the regulatory element.

Referring to the EP loop method, in various embodiments, identifying a candidate gene involves differentiating between whether the regulatory element is an enhancer or a promoter. In various embodiments, identifying a candidate gene involves identifying an enhancer-promoter loop (EP loop), where the enhancer and promoter of the EP loop are in contact with one another through a long range chromatin interaction. Therefore, a mutation in an activated enhancer that is located far from a gene can have an impact on the expression of the gene through the long range chromatin interaction.

In various embodiments, differentiating a regulatory element includes analyzing the protein-chromatin binding site pairing data (e.g., ChIP-seq data), the chromatin-accessibility data (e.g., ATAC-seq data), and sequencing/genotyping data (e.g., data identifying NCV). For example, for the position at which the NCV is located, a corresponding position in the protein-chromatin binding site pairing data is analyzed to determine whether a regulatory element-specific modification is present at the corresponding position. As a specific example, the protein-chromatin binding site pairing data may be H3K4me3 ChIP-seq data. Therefore, a presence of a peak in the protein-chromatin binding site pairing data would identify promoter-specific markers (e.g., H3K4me3) and therefore, presence of a promoter. A lack of a peak in the protein-chromatin binding site pairing data would indicate an absence of promoter-specific markers. Therefore, in a scenario where the chromatin-accessibility data includes a peak (e.g., indicating presence of a regulatory element) but the protein-chromatin binding site pairing data does not include a peak (e.g., indicating absence of promoter-specific marker), such an analysis reveals that the regulatory element at that position is an active enhancer.

In various embodiments, an active promoter is labeled based on presence of H3K4me3 peaks that colocalize with H3K27ac peaks. Furthermore, active enhancers are labeled based on presence of H3K27ac peaks that were outside of these active promoters. The presence of a peak in the ATAC-seq data, a peak in the H3K27Ac ChIP-seq data and a lack of peak in the H3K4me3 ChIP-seq data indicates a presence of an active enhancer.

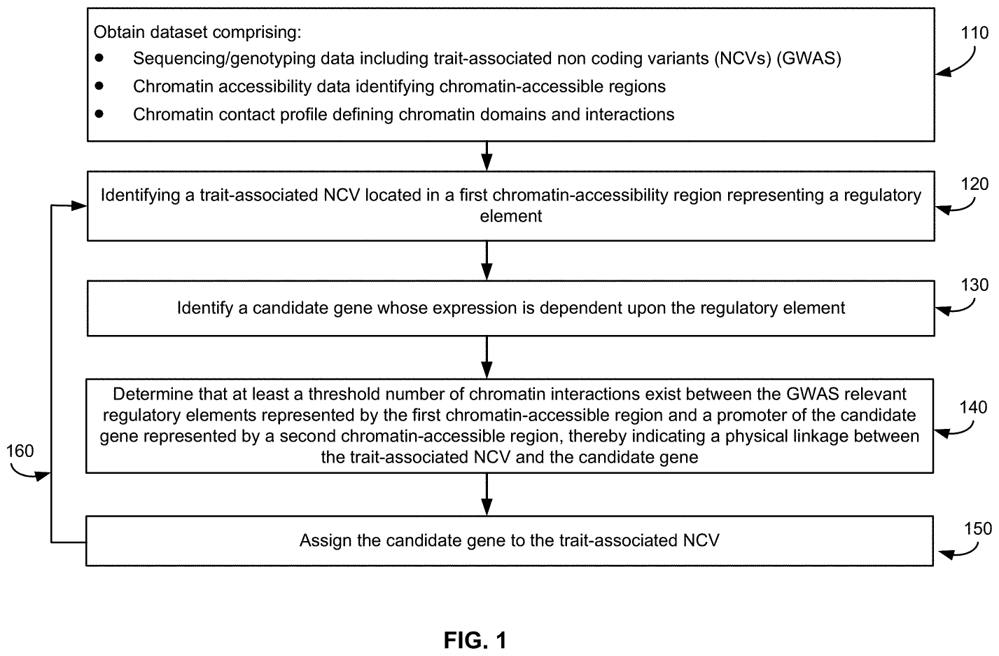

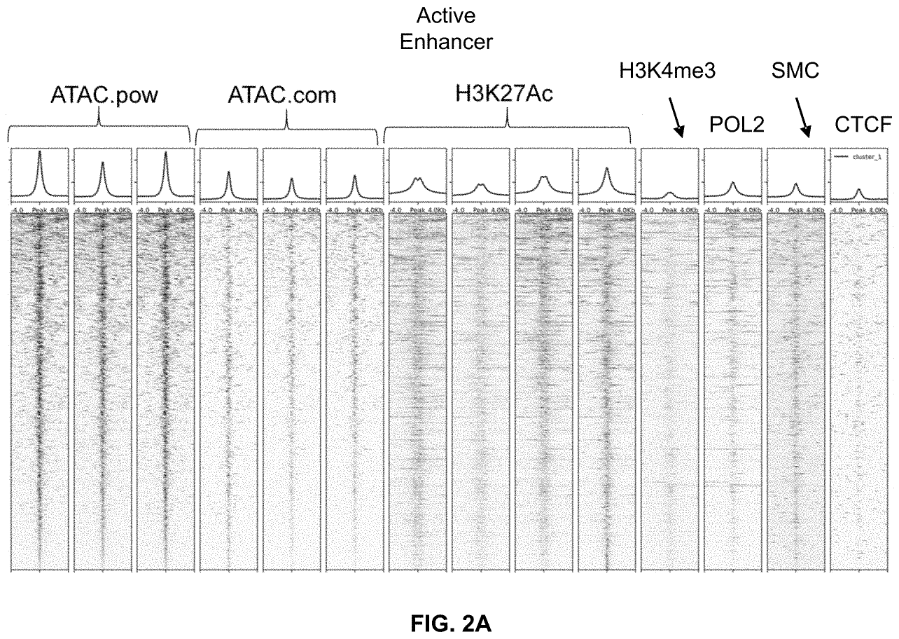

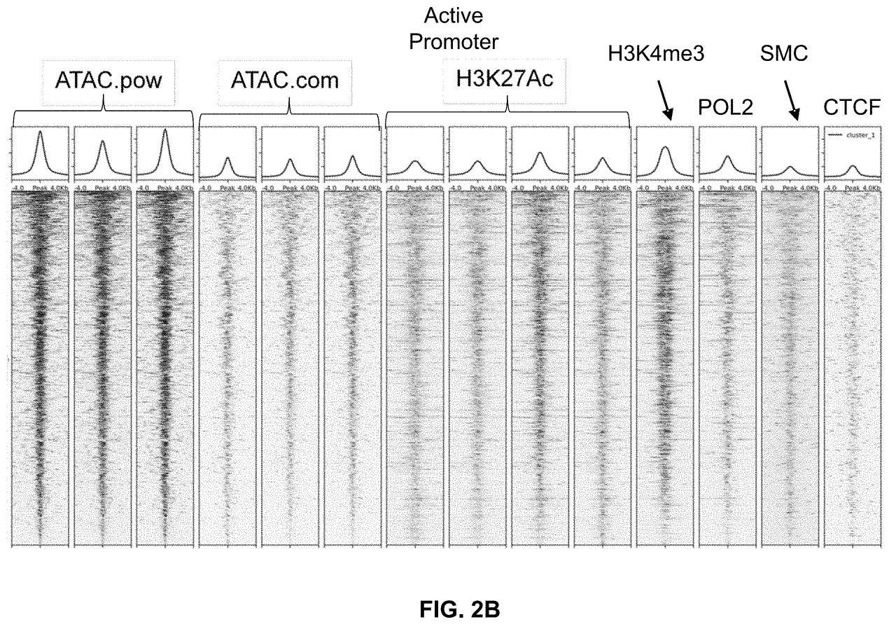

Reference is now made to FIGS. 2A and 2B, which depict example sets of data including chromatin-accessibility data and protein-chromatin binding site pairing data corresponding to an active enhancer and a promoter, respectively. ATAC.pow represents the ATAC-seq data in the primary human hepatocyte cultured in the power medium, while the ATAC.com represents the ATAC-seq data in the primary human hepatocyte cultured in the complete medium. Each of FIG. 2A and FIG. 2B, depicts data including chromatin-accessibility data in the form of ATAC-seq data (e.g., ATAC.pow and ATAC.com) and protein-chromatin binding site pairing data (e.g., H3K4me3). Additionally, the data in each of FIG. 2A and FIG. 2B include chromatin contact profile data as denoted by SMC1A, CTCF and H3K27Ac. For each of the chromatin accessibility data, protein-chromatin binding site pairing data, and chromatin contact profile data, a window corresponding to a range of positions are shown, thereby depicting either a presence or absence of a peak corresponding to the positions.

Referring first to FIG. 2A (presence of an active enhancer), the chromatin accessibility data (ATAC.pow and ATAC.com) include a peak, indicating presence of a regulatory element. Additionally, the protein-chromatin binding site pairing data (e.g., H3K4me3) does not include a substantive peak, thereby indicating absence of histones. Therefore, FIG. 2A shows example data corresponding to an active enhancer. Referring to FIG. 2B (presence of a promoter), the chromatin accessibility data (ATAC.pow and ATAC.com) include a peak, indicating presence of a regulatory element. Additionally, the protein-chromatin binding site pairing data (e.g., H3K4me3) also includes a peak, thereby indicating presence of histones. Therefore, FIG. 2B shows example data corresponding to an active promoter.

In various embodiments, an EP loop including an enhancer and a promoter is identified. For example, given the identification of regulatory elements as either an enhancer or a promoter, an EP loop can be identified using chromatin contact profile data (e.g., HiChIP), which defines chromatin domains and chromatin interactions (e.g., DNA-DNA interactions). These chromatin interactions can be mediated through proteins such as SMC1A, CTCF and H3K27Ac and therefore, can be identified through the chromatin contact profile data. The presence of peaks in the chromatin contact profile data corresponding to enhancers or promoters indicates chromatin contact (e.g., contact between an enhancer and a promoter). Thus, chromatin contact between an enhancer and a promoter indicates a possible presence of an EP loop.

In various embodiments, the candidate gene is identified through a promoter that is included in the EP loop. Here, the promoter in the EP loop is operably linked to the candidate gene, and furthermore, the enhancer in the EP loop governs the expression of the candidate gene. The identified candidate gene represents a gene whose expression is controlled by the regulatory elements of the EP loop and therefore, the expression of the identified candidate gene can be further affected by the presence of the non-coding variant.

Referring to the genetic association method, in various embodiments, the candidate gene is identified using the genetic variant (e.g., eQTL NCV) included in the LD block. The genetic variant (e.g., eQTL NCV) in the LD block is known to affect the expression of a gene. In various embodiments, the genetic variant can be known to affect the expression of a gene through a genotyping and gene expression data analysis from a group of samples. As a specific example, this analysis can be a meta-analysis of Genotype-Tissue Expression (GTEx) and additional other eQTL studies in the human liver. This analysis reveals associations of a genetic variant and gene expression. The gene may be in the same insulated neighborhood as the chromatin-accessible region (e.g., regulatory region) in the LD block. Thus, the gene associated with the genetic variant is deemed to be the candidate gene. In various embodiments, more than one genetic variant is included in the LD block. Therefore, more than one candidate gene is identified (e.g., a candidate gene is identified for each of the genetic variants in the LD block).

Step 140 involves determining a number of chromatin-chromatin interactions, based on the chromatin contact profile data, between the regulatory element represented by the first chromatin-accessible region and a location of a promoter of the candidate gene that is represented by a second chromatin-accessible region (as indicated by the chromatin accessibility data). The determined number of chromatin-chromatin interactions is compared to a threshold number of chromatin-chromatin interactions to ensure that the regulatory element and the promoter are located at a distance from each other. In various embodiments, the threshold number of chromatin-chromatin interactions is two, three, four, five, six, seven, eight, nine, or ten chromatin-chromatin interactions. In various embodiments, the threshold number of chromatin interactions is represented by a threshold number of HiChIP PETs detected by HiChIP-seq. Therefore, the threshold number of chromatin-chromatin interactions is represented by a number of HiChIP PETs underlying the chromatin-chromatin interactions. If the number of chromatin-chromatin interactions between the regulatory element and the promoter of the candidate gene is greater than the threshold number, then the regulatory element and the promoter of the candidate can be deemed physically linked through a long distance chromatin interaction. In some embodiments, the regulatory element and the promoter of the candidate gene can be linked in an insulated neighborhood.

At step 150, the candidate gene is assigned to the GWAS trait-associated NCV that was included in the LD block, thereby indicating that the GWAS trait-associated NCV likely impacts the expression of the candidate gene. Put another way, the GWAS trait-associated NCV is functionally annotated with the candidate gene.

In various embodiments, as indicated by 160, the steps of 120-150 can be repeated to identify additional linkages between non-coding variants and candidate genes. In one embodiment, identifying additional linkages between non-coding variants and candidate genes includes identifying additional associations non-coding variants and candidate genes through EP-loops. In one embodiment, identifying additional linkages between non-coding variants and candidate genes includes identifying additional LD blocks that include 1) a GWAS trait-associated NCV (as identified in the sequencing data) 2) a genetic variant, and 3) and a first chromatin-accessible region representing a regulatory element. Thus, candidate genes can be assigned to different GWAS trait-associated NCVs. In various embodiments, more than 2000 GWAS trait-associated NCVs can be functionally annotated with candidate genes.

In various embodiments, the linkage between a GWAS trait-associated NCV and a candidate gene can be leveraged to identify therapeutics for disease treatment. For example, the GWAS trait-associated NCV may be linked to a candidate gene that is known to be associated with a disease. Therefore, targeting the regulatory element (e.g., enhancer) of the candidate gene can be a method of treating the disease.

In one embodiment, a target protein that affects the regulatory element (e.g., binds to the regulatory element) can be identified using protein-chromatin binding site data and/or transcription factor binding motif data. In various embodiments, the target protein is identified by using the protein-chromatin binding site data and transcription factor binding motif data located between the ATAC-peaks representing the location of the regulatory element and the location of the promoter of the candidate gene. For example, the protein-chromatin binding site data may identify the particular binding site that corresponds to the regulatory element and additionally, may identify the transcription factor that binds to the regulatory element. This transcription factor can be a target protein that, if targeted, can modulate the expression of the candidate gene. As another example, the transcription factor binding motif data may be used to identify a particular motif corresponding to the regulatory element. A protein that binds to the particular motif can be identified and therefore, can serve as the target protein.

In various embodiments, the target protein is used to identify a signaling pathway such that additional targets can be identified. For example, the target protein may be bound by a receptor that is involved in the signaling pathway. Therefore, additional proteins and/or receptors involved in the signaling pathway can serve as a possible target. For example, the receptor of the target protein can also be a target such that, if targeted, the altered expression of the receptor would also modulate the expression of the candidate gene.

In various embodiments, a therapeutic can be selected. In some embodiments, the therapeutic is previously known to modulate expression of the target protein (e.g., a transcription factor that binds to the enhancer and regulates expression of a candidate gene). For example, the therapeutic may serve to knockdown or upregulate the expression of the target protein, which, as a result, would serve to modulate the expression of the candidate gene. In some embodiments, the therapeutic is previously known to modulate expression of a receptor or additional protein involved in the identified signaling pathway. The therapeutic can serve to knockdown or upregulate the expression of a receptor or additional protein, thereby serving to modulate the expression of the candidate gene.

II. Example Computer

The methods described above, such as the methods of identifying a therapeutic target by discovering interactions between trait-associated NCVs and candidate genes, are, in some embodiments, performed on a computer.

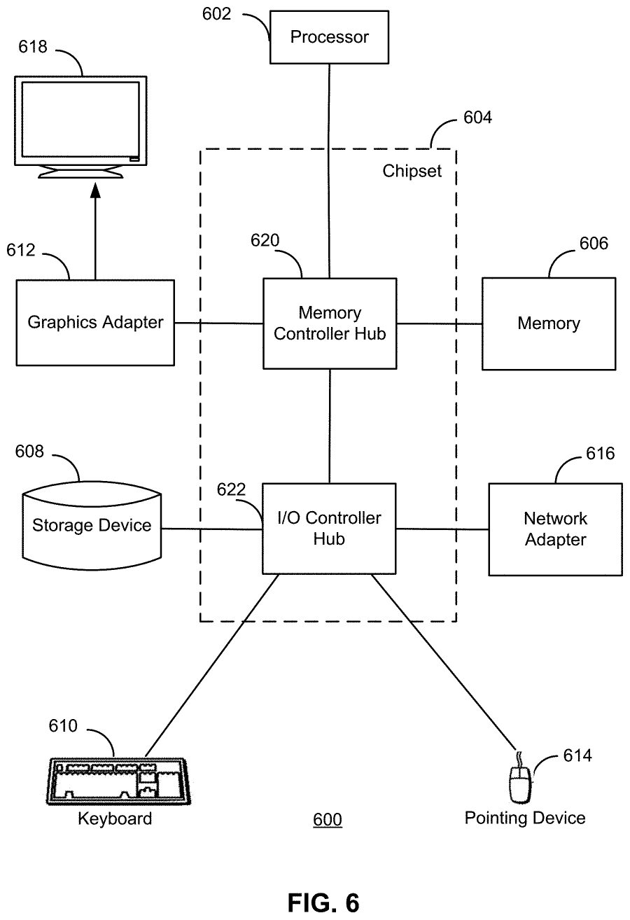

FIG. 6 illustrates an example computer 600 for implementing the steps of the flow diagram shown in FIG. 1. The computer 600 includes at least one processor 602 coupled to a chipset 604. The chipset 604 includes a memory controller hub 620 and an input/output (I/O) controller hub 622. A memory 606 and a graphics adapter 612 are coupled to the memory controller hub 620, and a display 618 is coupled to the graphics adapter 612. A storage device 608, an input device 614, and network adapter 616 are coupled to the I/O controller hub 622. Other embodiments of the computer 600 have different architectures.

The storage device 608 is a non-transitory computer-readable storage medium such as a hard drive, compact disk read-only memory (CD-ROM), DVD, or a solid-state memory device. The memory 606 holds instructions and data used by the processor 602. The input interface 614 is a touch-screen interface, a mouse, track ball, or other type of pointing device, a keyboard, or some combination thereof, and is used to input data into the computer 600. In some embodiments, the computer 600 may be configured to receive input (e.g., commands) from the input interface 614 via gestures from the user. The graphics adapter 612 displays images and other information on the display 618. The network adapter 616 couples the computer 600 to one or more computer networks.

The computer 600 is adapted to execute computer program modules for providing functionality described herein. As used herein, the term “module” refers to computer program logic used to provide the specified functionality. Thus, a module can be implemented in hardware, firmware, and/or software. In one embodiment, program modules are stored on the storage device 608, loaded into the memory 606, and executed by the processor 602.

The types of computers 600 that are used to implement the steps of the flow diagram shown in FIG. 1 can vary depending upon the embodiment and the processing power required. For example, the flow diagram shown in FIG. 1 can be executed by a single computer 600 or across multiple computers 600 communicating with each other through a network such as in a server farm. The computers 600 can lack some of the components described above, such as graphics adapters 612, and displays 618.

III. Non-transitory Computer Readable Medium

Also provided herein is a computer readable medium comprising computer executable instructions configured to implement any of the methods described herein. In various embodiments, the computer readable medium is a non-transitory computer readable medium. In some embodiments, the computer readable medium is a part of a computer system (e.g., a memory of a computer system). The computer readable medium can comprise computer executable instructions for implementing the method described above to identify therapeutic targets by discovering interactions between trait-associated NCVs and candidate genes.

### EXAMPLES

### Example 1: Method for Identifying Therapeutic Targets by Discovering Long Range Chromatin Interactions Between NCVs and Candidate Genes

A method for identifying long range chromatin interactions between NCVs and candidate genes is described below.

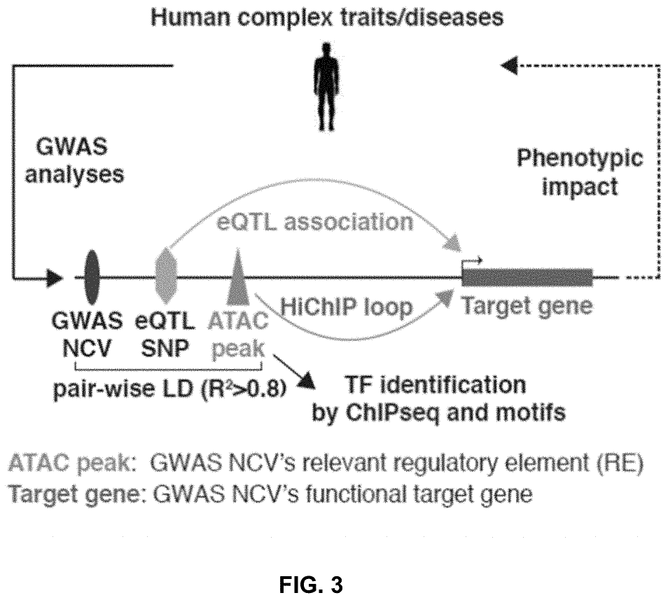

FIG. 3 depicts an example diagram for identifying interactions between a non-coding variant and a target gene. A dataset was obtained including 1) GWAS data (e.g., sequencing or genotyping data) which included a non-coding variant (NCV), 2) ATAC-seq data (e.g., chromatin accessibility data) identifying chromatin-accessible regions across the genome; 3) ChIP-seq data (e.g., protein-chromatin binding site data), and 4) HiChIP data (e.g., chromatin contact profile) describing chromatin-chromatin interactions and chromatin domain. Additional data included Expression Quantitative Trait Loci (eQTL) data including eQTL single nucleotide polymorphisms (eQTL SNPs) and pairwise linkage disequilibrium (LD) data derived from over 1000 Genomes.

Further description of the example data in the dataset is detailed below in Tables 1 and 2.

TABLE 1
The following data was obtained by performing the corresponding
techniques (shown in parenthesis) in primary human
hepatocytes obtained from multiple donors.

| Data (Technique) | Description of Data |
|---|---|
| Chromatin contact | Defines chromatin domains and chromatin inter- |
| profile data | actions (e.g., DNA-DNA interactions) mediated by |
| (HiChIP) | SMC1A, CTCF and H3K27Ac in 4kb resolution |
| Chromatin | Elucidates DNA-protein interaction sites for |
| accessibility data | transcription factors and chromatin binding proteins |
| (ATAC-seq) | |
| Protein-chromatin | Specifically reveals binding sites of > 60 |
| binding site data | transcription factors, chromatin binding proteins |
| (ChIP-seq) | and histone modifications |
| Gene expression | Quantifies gene expression changes in response |
| data (RNA-seq) | to > 900 perturbagens (compounds, ligand, siRNAs) |

TABLE 2
The following published datasets were used in the analysis
for identifying long range chromatin interactions.

| Published datasets | Description of Data |
|---|---|
| Genome Wide | ~84k complex trait associated single nucleotide |
| Association Study | polymorphisms (SNPs) from GWAS catalog |
| (GWAS) | |
| Expression | ~830k associations from a meta analysis of |
| Quantitative Trait | Genotype-Tissue Expression (GTEx) and three other |
| Loci (eQTL) | eQTL studies in the human liver |
| Pairwise linkage | Pairwise linkage disequilibrium (LD) measurements |
| disequilibrium | based on the 1000 Genomes |
| (LD) data | |
| Transcription factor | 1639 motifs from the Human Transcripton Factor |
| binding motifs | motifs database |

Linkage disequilibrium blocks were obtained from the pairwise LD data that were associated with a square of a correlation coefficient above 0.8 (R²>0.8). LD blocks were generated using Plink, which measures the pairwise LD for multiple genome-wide SNPs. Linkage disequilibrium blocks were analyzed to determine which LD blocks included 1) a GWAS non-coding variant (from the GWAS dataset), 2) an eQTL variant (from the eQTL dataset), and 3) an ATAC-peak (from ATAC-seq data).

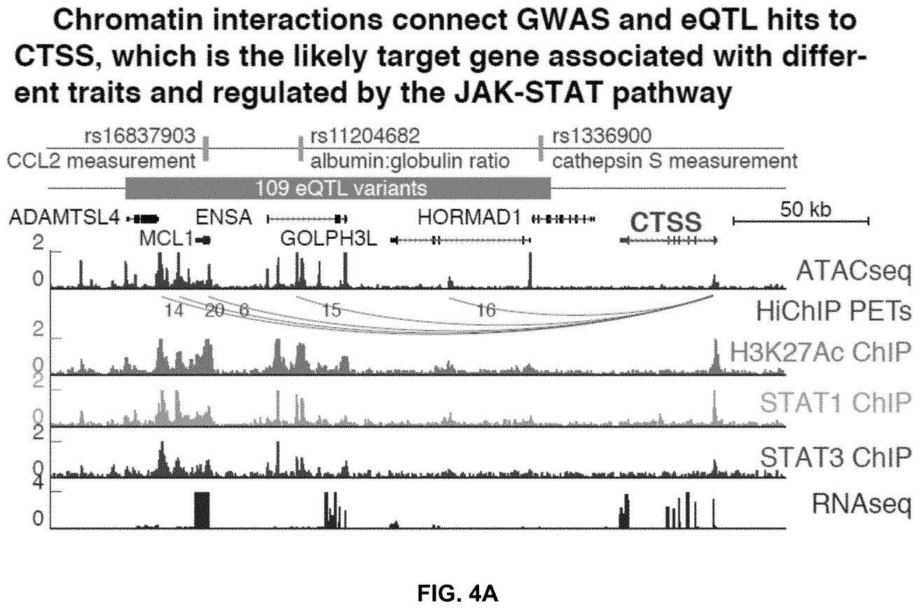

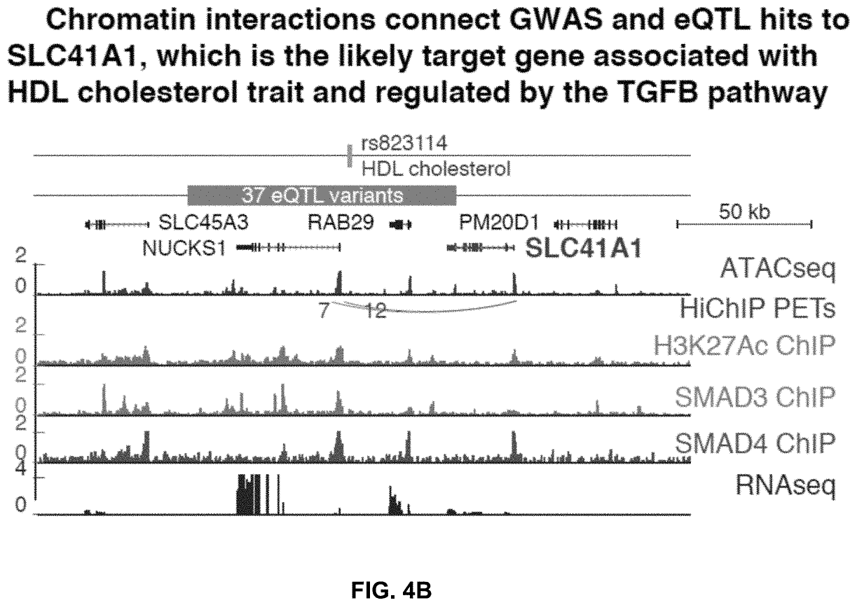

For the identified LD blocks that included the three elements described above, a candidate gene was identified. Here, the eQTL variant in the eQTL dataset is known to be associated with a gene (e.g., the eQTL is known to affect the expression of the gene). Therefore, the gene associated with the eQTL variant is identified as a candidate gene for the LD block. Two example candidate genes were identified through this method which include CTSS and SLC41A1. FIG. 4A depicts the example linkage between a non-coding variant and a target gene CTSS. FIG. 4B depicts the example linkage between a non-coding variant and a target gene SLC41A1.

The ATAC peak in the LD block was taken to represent a regulatory element, such as an enhancer. The location of a promoter of the candidate gene was determined based on a location of a second ATAC peak in the ATAC-seq. Next, an analysis focused on the chromatin region between 1) the regulatory element, represented by the ATAC-peak in the LD block, and 2) the location of the promoter of a candidate gene. Specifically, the number of chromatin-chromatin interactions in this chromatin region was calculated using the HiChIP-seq data that included HiChIP paired-end tags (PETs). The number of HiChIP PETs was compared to a threshold number of 5. If the number of HiChIP PETs was above 5, then the regulatory element and the promoter of the candidate gene was deemed to be physically linked through a long range chromatin interaction (e.g., in an insulated neighborhood) (e.g., shown as HiChIP loop in FIG. 3).

The candidate gene was then assigned to the GWAS non-coding variant (NCV) included in the LD block that also included the ATAC peak representing the physically linked regulatory element. In total, 2870 GWAS NCVs were functionally annotated in the primary human hepatocyte by identifying relevant regulatory elements (2087) and target genes (1164).

This link between a candidate gene and GWAS NCV was then used to identify therapeutic targets. Using protein-chromatin binding site data (e.g., obtained through ChIP-seq) and the transcription factor binding motif data, proteins that bind to the regulatory element represented by the ATAC-peak in the LD block were identified. For example, STAT1 was identified as a protein that bound with a relevant regulatory element for the CTSS gene. Additionally, SMAD3 was identified as a protein that bound with a relevant regulatory element for the SLC41A1 gene.

The identified protein was used to determine a signaling pathway that the protein was involved in. For example, the STAT1 protein was used to identify the JAK-Stat signaling pathway. Altogether, 246 GWAS target genes were identified as being linked to a regulatory element through the JAK-Stat signaling pathway. Similarly, the SMAD3 protein was used to identify the TGFB pathway. Altogether, 462 GWAS target genes were identified as being linked to a regulatory element through the transforming growth factor beta (TGFB) signaling pathway.

With knowledge of a signaling pathway, a protein or a receptor involved in the signaling pathway was identified to serve as a therapeutic target that can be modulated to alter the expression of the gene. For example, knockdown of a receptor (e.g., interferon gamma (IFNG)) or a protein (e.g., STAT1) involved in the JAK-Stat signaling pathway was shown to modulate the expression level of the CTSS gene. Similarly, knockdown of TGFB1 and SMAD3 modulated the expression level of the SLC41A1 gene.

### Example 2: Example Connections Between Trait-Associated Non-Coding Variants and Candidate Genes

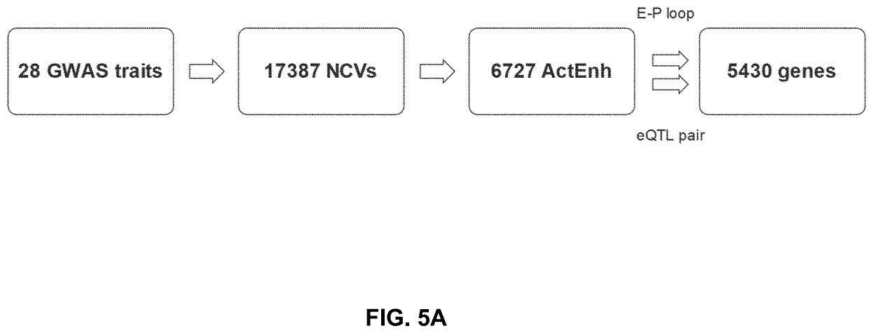

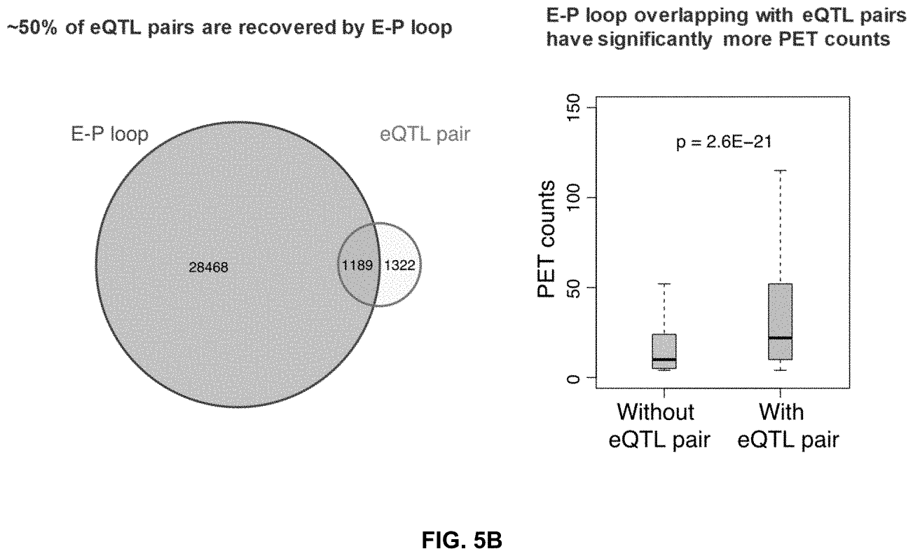

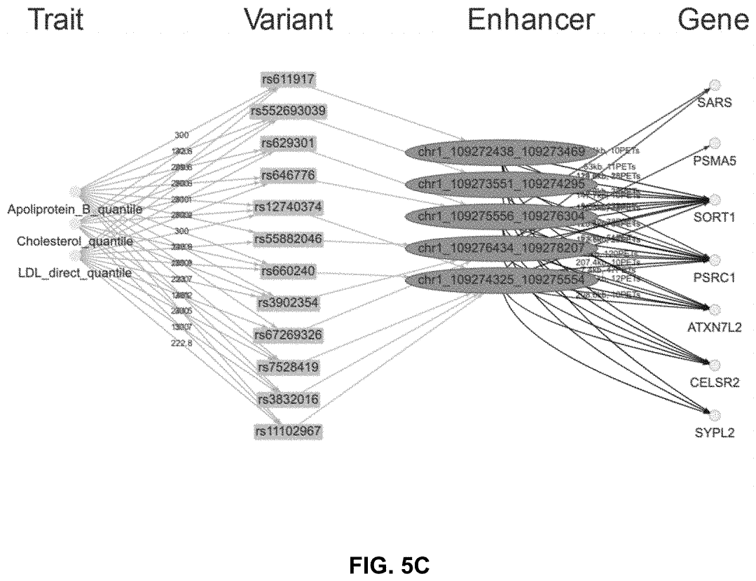

FIG. 5A depicts an overall pipeline of genes that were linked to activated enhancers using the methods described herein. In total, 28 of 1188 traits from UK Biobank (UKBB) GWAS study were functionally associated with the hepatocyte based on partitioning heritability analysis. The 28 traits included gamma glutamyltransferase, alanine aminotransferase, aspartate aminotransferase, creatinine, alkaline phosphatase, urate, IGF-1, cystatin-C, SHBG, albumin, triglycerides, apolipoprotein B, haemoglobin concentration, HDL cholesterol, phosphate, total bilirubin, apolipoprotein A, immature reticulocyte, LDL concentration, cholesterol, urea, vitamin D, testosterone, glycated haemoglobin, rheumatoid arthritis, disorders of gallbladder biliary tract and pancreas, c-reactive protein, and calculus of kidney and ureter. 17,387 non-coding variants (NCVs) were identified from the study. The NCVs were associated with 6727 activated enhancers. Using the methods described herein (e.g., E-P loop method and genetic association method (“eQTL pair”)), a total of 5430 genes were identified and assigned to NCVs.

FIG. 5B depicts overlapping non-coding variant and gene pairs using the EP loop method or the genetic association method. Specifically, FIG. 5B shows a comparison between the EP loop method and the genetic association method (eQTL pair method). The EP loop method identified over 28,000 pairs of NCVs and candidate genes whereas eQTL identified associations of 2500 pairs of NCVs and candidate genes. Of note, ˜50% of eQTL pairs (e.g., 1189 pairs) were similarly found through the EP loop method. Additionally, of these 1189 pairs that are identified through both E-P loop and eQTL pair method, significantly more PET counts are observed. This indicates that either the EP loop method or the eQTL pair method can be suitable for identifying pairs of NCVs and candidate genes. Additionally, the combination of EP loop method and eQTL pair method may enable the identification of longer range chromatin interactions, as indicated by the significantly more PET counts.

FIG. 5C depicts examples linkages between trait-associated non-coding variants and target genes. As shown in FIG. 5, the different non-coding variants are further associated with different traits, such as liver-related traits (e.g., apolipoprotein B, cholesterol, and low-density lipoproteins (LDL). Here, linkages between the non-coding variants and the genes were determined using the method described above in FIG. 1. Specifically, a non-coding variant was determined to be located in a region representing an active enhancer. Here, an active enhancer was determined using ATAC-seq data (representing chromatin accessible regions) and ChIP-seq data (protein-chromatin binding site pairing data). The presence of a peak in the ATAC-seq data, a peak in the H3K27Ac ChIP-seq data and a lack of peak in the H3K4me3 ChIP-seq data indicates a presence of an active enhancer. A candidate gene whose expression is dependent upon the active enhancer is then identified.

Next, the number of chromatin-chromatin interactions in this chromatin region was calculated using the HiChIP-seq data that included HiChIP paired-end tags (PETs). The number of HiChIP PETs was compared to a threshold number of 5. If the number of HiChIP PETs was above 5, then the active enhancer and a promoter of the candidate gene was deemed to be physically linked through a long range chromatin interaction (e.g., in an insulated neighborhood). Thus, the non-coding variant is linked to the candidate gene (e.g., through the active enhancer).

1. A method, comprising:
obtaining a dataset comprising:
sequencing or genotyping data comprising a non-coding variant (NCV);
chromatin accessibility data identifying chromatin-accessible regions across the genome; and
chromatin contact profile describing chromatin-chromatin interactions and chromatin domains;
identifying that the NCV is located in a first chromatin-accessibility region representing a regulatory element;
identifying a candidate gene whose expression is dependent upon the regulatory element;
determining that at least a threshold number of chromatin-chromatin interactions exist between a location of the first chromatin-accessible region representing the regulatory element and the location of the second chromatin-accessible region representing a promoter of the candidate gene, thereby indicating that the promoter of the candidate gene and the regulatory element are physically linked through a long range chromatin interaction; and
linking the candidate gene to the NCV.

2. The method of claim 1, wherein the obtained dataset further comprises protein-chromatin binding site pairing data, and wherein identifying that the NCV is located in a first chromatin-accessibility region representing a regulatory element comprises determining that the regulatory element is positioned at the first chromatin-accessibility region using the protein-chromatin binding site pairing data.

3. The method of claim 2, wherein the regulatory element is an active enhancer.

4. The method of claim 3, wherein determining that the regulatory element is positioned at the first chromatin-accessibility region using the protein-chromatin binding site pairing data comprises identifying the regulatory elements as an active enhancer as opposed to a promoter by comparing the chromatin accessibility data to the protein—chromatin binding site pairing data.

5. The method of claim 4, wherein the chromatin accessibility data corresponding to the regulatory element comprises a peak indicating presence of a regulatory element, and wherein the protein-chromatin binding site pairing data corresponding to the regulatory element lacks a peak, thereby indicating lack of a promoter.

6. The method of claim 1, wherein identifying a candidate gene whose expression is dependent upon the regulatory element comprises:
determining a location of a promoter of the candidate gene based on a location of a second chromatin-accessible region in the chromatin accessibility data; and
constructing an enhancer-promoter (E-P) loop comprising the regulatory element and the promoter of the candidate gene.

7. The method of claim 1, wherein the dataset further comprises a genetic variant, wherein identifying a candidate gene whose expression is dependent upon the regulatory element comprises identifying the candidate gene as a gene whose expression is affected by the genetic variant.

8. The method of claim 7, wherein the genetic variant is selected from any one of an expression quantitative trait loci (eQTL) variant determined from a meta-analysis of Genotype Tissue Expression (GTEx) data or a source of gene expression profile data measured with DNA sequence variation data.

9. The method of claim 2, wherein the protein-chromatin binding site pairing data comprises ChIP-seq data.

10. The method of claim 1, wherein the sequencing or genotyping data comprising the non-coding variant (NCVs) is Genome-Wide Association Study (GWAS) data.

11. The method of claim 1, wherein the sequencing or genotyping data comprising the non-coding variant (NCVs) is obtained from whole genome sequencing, whole exome sequencing, targeted of a gene region or panel of genes, or targeted genotyping.

12. The method of claim 1, wherein the chromatin accessibility data is Assay for Transposase-Accessible Chromatin (ATAC) sequencing data comprising ATAC peaks that identify the chromatin-accessible regions across the genome.

13. The method of claim 1, wherein the chromatin contact profile is HiChIP-seq data comprising HiChIP paired-end-tags (PETs) describing the chromatin-chromatin interactions.

14. The method of claim 1, wherein the threshold number of chromatin-chromatin interactions is represented by three, four, five, six, seven, or eight HiChIP paired-end-tags.

15. The method of claim 1, wherein at least one of the chromatin-chromatin interactions of the chromatin contact profile is an enhancer-promoter interaction.

16. The method of claim 1, wherein at least one of the chromatin domains of the chromatin contact profile is an insulated neighborhood.

17. The method of claim 1, wherein the dataset further comprises transcription factor binding motif data, wherein the method further comprises:
using the transcription factor binding motif data, identifying a target protein that binds at the location of the first chromatin-accessible region representing the regulatory element;
determining a signaling pathway that the target protein is involved in;
selecting one or more therapeutics capable of modulating the signaling pathway which would subsequently modulate expression of the candidate gene.

18. The method of claim 17, wherein using the transcription factor binding motif data comprises identifying transcription factor binding motif data corresponding to sequences located between the first chromatin-accessible region and the second chromatin-accessible region.

19-35. (canceled)

36. A non-transitory computer readable medium comprising instructions that, when executed by a processor, cause the processor to:
obtain a dataset comprising:
sequencing or genotyping data comprising a non-coding variant (NCV);
chromatin accessibility data identifying chromatin-accessible regions across the genome; and
chromatin contact profile describing chromatin-chromatin interactions and chromatin domains;
identify that the NCV is located in a first chromatin-accessibility region representing a regulatory element;
identify a candidate gene whose expression is dependent upon the regulatory element;
determine that at least a threshold number of chromatin-chromatin interactions exist between a location of the first chromatin-accessible region representing the regulatory element and the location of the second chromatin-accessible region representing a promoter of the candidate gene, thereby indicating that the promoter of the candidate gene and the regulatory element are physically linked through a long range chromatin interaction; and
link the candidate gene to the NCV.

37. The non-transitory computer readable medium of claim 36, wherein the dataset further comprises protein-chromatin binding site pairing data, and wherein the instructions that cause the processor to identify that the NCV is located in a first chromatin-accessibility region representing a regulatory element further comprises instructions that, when executed, cause the processor to determine that the regulatory element is positioned at the first chromatin-accessibility region using the protein-chromatin binding site pairing data.

38-69. (canceled)
